(19) United States
(12) Patent Application Publication
(10) Pub. No.: US 2008/0233082 A1
(43) Pub. Date: Sep. 25, 2008

(54) POLYMER WITH ABILITY TO SIGNAL THE RECRUITMENT OF VASCULAR PROGENITOR CELLS

(75) Inventors: Brian Hoh, Gainesville, FL (US); Chris Batich, Gainesville, FL (US); Edward Scott, Gainesville, FL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Florida

(21) Appl. No.: 12/051,188

(22) Filed: Mar. 19, 2008

Related U.S. Application Data
(60) Provisional application No. 60/895,827, filed on Mar. 20, 2007.

(51) Int. Cl. A61K 38/20; A61F 2/82; A61K 38/48; A61P 9/00; A61K 38/16

(52) U.S. Cl. 424/85.2; 623/1.42; 424/94.67; 514/12

(57) ABSTRACT

The present invention provides polymeric compositions, devices, and methods for repair of vascular injury, particularly endothelial injury and impaired vascular repair, such as from intracranial aneurysms and carotid atherosclerosis. The invention particularly provides polymers incorporating signaling factors useful for recruiting circulating vascular progenitor cells. The polymers can be used alone or as a coating for various devices for placement at sites of vascular injury to promote repair by the body's natural systems.

### CROSS-REFERENCE TO RELATED APPLICATIONS

The present application claims priority to U.S. Provisional Patent Application No. 60/895,827, filed Mar. 20, 2007, which is incorporated herein by reference in its entirety.

### FIELD OF THE INVENTION

The present invention is related to polymers useful for vascular repair. More particularly, the invention is related to polymers capable of signaling vascular progenitor cell activity for vascular repair.

### BACKGROUND

Aneurysmal subarachnoid hemorrhage (SAH), or bleeding between the middle membrane covering of the brain and the brain itself (the subarachnoid space), is a devastating event and occurs in approximately 30,000 Americans each year. Though relatively less frequent than other stroke subtypes, SAH typically affects a younger population and results in 30% to 50% mortality. Further, survivors of SAH exhibit 50% significant morbidity. The economic healthcare costs associated with SAH are enormous. The lifetime economic healthcare cost of an individual in the United States affected by SAH has been calculated to be $228,030 (the highest among all stroke subtypes); the aggregate lifetime costs for all individuals affected by SAH in the United States in one year has been calculated to be $5.6 billion.

Current non-surgical therapy for aneurysms includes endovascular coil embolization. For such treatment, a catheter is typically guided through the femoral artery into the brain vessels and into the aneurysm where soft platinum coils are deposited. When in position, the coil is released, such as by an application of a very low voltage current. The softness of the platinum allows the coil to conform to the often irregular shape of an aneurysm. On average, 5-10 coils are usually required to completely pack an aneurysm. The goal of this treatment is to prevent blood flow into the aneurysm sac by filling the aneurysm with coils and thrombus, which is intended to prevent aneurysm bleeding or re-bleeding.

A multicenter prospective randomized trial comparing the surgical treatment of aneurysms to endovascular coil embolization in subarachnoid hemorrhage patients demonstrated a significant clinical benefit to endovascular therapy (30.9% one-year morbidity and mortality in the surgical patients compared to 23.5% in the endovavascular patients). A major drawback in endovascular coil therapy for aneurysms is aneurysm recurrence after treatment. Aneurysm recurrence after coil embolization occurs in 21-34% of coil-embolized aneurysms. The formation of neo-endothelium across the orifice of the coil-treated aneurysm seems to be critical in preventing aneurysm recurrence. Improvement in vascular repair and endothelialization after coil embolization would reduce the incidence of aneurysm recurrence and significantly enhance the results and clinical benefit to patients with endovascular therapy for aneurysms.

Another vascular condition associated with serious health problems is carotid atherosclerosis. Every year approximately 700,000 people in the United States suffer a new or recurrent stroke. Stroke is the third leading cause of death and the leading cause of disability in the United States. In 2006, the estimated direct and indirect cost of stroke was approximately $57.9 billion, and the mean lifetime cost of ischemic stroke is estimated at $140,048. As many as 20% of strokes are due to carotid artery disease, and carotid artery stenosis has been shown to be highly correlated with myocardial infarction and stroke.

Surgical treatment for carotid atherosclerosis typically comprises surgical removal of plaque from the artery (endarterectomy). Current non-surgical therapy for carotid atherosclerosis includes percutaneous balloon angioplasty and intravascular stent placement. In a multicenter prospective randomized trial comparing carotid endarterectomy to carotid angioplasty and stenting, there were significantly better clinical outcomes in the patients treated with angioplasty and stenting (20.1% one-year morbidity and mortality in the endarterectomy patients compared to 12.2% in the angioplasty and stenting patients).

A major drawback in carotid angioplasty and stenting is in-stent restenosis (i.e., the recurrence of stenosis), which occurs in as many as 16% of patients. The establishment of an intact endothelium after stent placement is critical to preventing restenosis and thrombosis. Improvement in vascular repair and endothelialization after carotid angioplasty and stenting would reduce the incidence of carotid restenosis and significantly enhance the results and clinical benefit to patients with carotid atherosclerotic disease.

Bone-marrow derived circulating vascular progenitor cells have been shown to migrate to sites of vascular injury. These circulating vascular progenitor cells, rather than local neighboring cells, seem to be the agents for vascular repair. The signaling pathways for the recruitment of circulating vascular progenitor cells have been studied but are not entirely elucidated. Thus, it would be useful to have mechanism for promoting the recruitment of circulating vascular progenitor cells and thus improving the treatment of vascular conditions, such as those described above.

### SUMMARY OF THE INVENTION

Vascular progenitor cells are believed to circulate in the blood and migrate to the sites of vascular injury to perform vascular repair. When vascular repair is impaired, atherosclerosis and aneurysms develop. The present invention takes advantage of the vascular progenitor cells naturally present in the blood to promote and enhance vascular repair. In particular, the present invention utilizes polymeric materials that have been specifically modified to signal the recruitment of vascular progenitor cells and enhance their ability for vascular repair. In specific embodiments, the polymeric materials incorporate specific factors that signal such recruitment of vascular progenitor cells. Thus, the invention allows for harnessing the power of the circulating vascular progenitor cells to perform vascular repair and even create new vessel walls at sites of vascular injury.

In one aspect, the present invention provides compositions useful in the repair of vascular injury. Such compositions are useful in treatment of injury to blood vessels generally. In certain embodiments, the composition comprises a polymer incorporating useful signaling factors. In specific embodiments, the signaling factors comprising factors that signal the recruitment of vascular progenitor cells. Preferentially, the polymer used according to the invention comprises a biodegradable polymer. In specific embodiments, the polymer comprises poly(lactide-co-glycolide), also known as PLGA. Of course, other types of polymers could be used and are specifically encompassed by the present invention.

The compositions of the invention are particularly characterized in that they physically function to signal the recruitment of vascular progenitor cells and enhance their ability for vascular repair. Preferably, the inventive polymers are modified to incorporate one or more factors that naturally signal such recruitment. In certain embodiments, the factors incorporated into the polymer comprise one or more chemokines. In specific embodiments, the signaling factors are selected from the group consisting of stromal cell-derived factor-1, P-selectin, E-selectin, L-selectin, E-NOS, I-NOS, ICAM, VCAM, VEGF, CXCR4, matrix metalloproteinases (MMPs), CTGF, angiogenin, angiopoietin-1, del-1, fibroblast growth factors (FGFs), follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, insulin-like growth factors (IGFs), and combinations thereof.

The polymer composition of the invention can be used by itself for vascular repair or may be incorporated in, or included with, another treatment, such as a medical device. Accordingly, in another aspect, the present invention provides devices useful in vascular repair. For example, in some embodiments, the invention is directed to a device for use in vascular repair, wherein the device comprises a polymer incorporating signaling factors that signal the recruitment of vascular progenitor cells. Specifically, the devices according to the invention can comprise a variety of devices, including stents (e.g., coronary stents, peripheral stents, carotid stents, intracranial stents, and aortic stent grafts) and aneurysm coils. For example, the polymer could be used to at least partially coat a pre-made stent or aneurysm coil.

In further embodiments, the polymer of the invention can be used to form a device useful in the treatment of vascular injury. The device can be comprises partially or totally from the polymer composition of the invention. For example, the polymer can be used to form a device, such as a scaffold, to be placed within a damaged vessel or artery. In specific embodiments, the polymer could be used by itself to form a stent or a coil. All of the devices according to the invention are particularly useful in that they can easily be delivered in the patient endovascularly. Of course, the devices could also be delivered by one or more surgical techniques. Thus, the devices provided according to the invention can be described as intravascular devices.

The variety of compositions and devices possible according to the invention are particularly useful in that they allow for many types of beneficial therapeutic uses. For example, in one embodiment of the invention, the compositions and devices are useful in a method for signaling the recruitment the vascular progenitor cells at a site of vascular injury. The invention is particularly useful in that it can be used in blood vessels generally and can be used to treat a variety of vascular injuries. In preferred embodiments, the method comprises providing at the site of vascular injury a composition comprising a polymer incorporating signaling factors that signal the recruitment of vascular progenitor cells. Similarly, in a further embodiment, the compositions and devices are useful in a method of enhancing vascular repair at a site of vascular injury. Preferentially, the method comprises providing at the site of vascular injury a composition comprising a polymer incorporating signaling factors that signal the recruitment of vascular progenitor cells.

In yet further embodiments, the invention provides compositions comprising a polymer, as described herein, and vascular progenitor cells. The vascular progenitor cells can be combined with the polymer in a number of fashions, such as being mixed throughout the polymer. In one embodiment, the polymer is formed into a desired shape having an outer surface, and the vascular progenitor cells are on at least a portion of the outer surface of the polymer. The compositions of the invention comprising the vascular progenitor cells can further comprise signaling factors that signal the recruitment of vascular progenitor cells.

### DETAILED DESCRIPTION OF THE INVENTION

The present inventions now will be described more fully hereinafter with reference to specific embodiments of the invention. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

As previously pointed out, current therapies for atherosclerosis (particularly the use of stents) are plagued by high incidence of restenosis. Likewise, current therapies for aneurysms (particularly the use of aneurysm coils) are plagued by aneurysm recurrence. The present invention solves these problems by providing polymeric compositions and devices incorporating or including such polymeric compositions that promote and enhance vascular repair. Moreover, the invention provides compositions and devices that can be used in blood vessels generally and can be used to treat a wide variety of vascular injuries.

Many vascular pathologic conditions are triggered by endothelial disruption that arises when the body's mechanisms for vascular repair go awry. It was once thought that vascular repair was a local phenomenon involving neighboring vascular cells; however, it has now been demonstrated that the critical agents for vascular repair are bone marrow-derived circulating vascular progenitor cells. When sufficient circulating vascular progenitor cells can migrate and attach to a site of vascular injury, they can mount an adequate vascular repair process and re-establish an intact endothelium. One particular problem solved by the present invention is overcoming inherent limits on the amount of vascular progenitor cells available to stimulate vascular repair.

The signaling pathways for recruiting vascular progenitor cells to the site of vascular injury seem to particularly involve chemokines, a group of structurally related proteins that participate in mechanisms of leukocyte migration. Proteins are classified as chemokines according to shared structural characteristics such as small size (they are all approximately 8-10 kilodaltons in size), and the presence of four cysteine residues in conserved locations that are key to forming their 3-dimensional shape. Their name is derived from their ability to induce directed chemotaxis in nearby responsive cells (i.e., they are chemotactic cytokines). These proteins have historically been known under several other names including the SIS family of cytokines, SIG family of cytokines, SCY family of cytokines, Platelet factor-4 superfamily, or intercrines. Some chemokines are homeostatic and are involved in controlling the migration of cells during normal processes of tissue maintenance or development. Chemokines are found in all vertebrates, some viruses, and some bacteria. These proteins exert their biological effects by interacting with G protein-linked transmembrane receptors called chemokine receptors that are selectively found on the surfaces of their target cells.

While chemokines are particularly believed to be involved in signaling the recruitment of vascular progenitor cells, other signaling factors may also be involved in the process. Accordingly, any signaling factor that functions to stimulate or enhance recruitment of vascular progenitor cells at a site of vascular injury could be used according to the present invention. For example, stromal cell-derived factor-1 (SDF-1), also known as pre-B cell growth-stimulating factor, is produced by bone marrow stromal cells and acts together with interleukin-7 as a co-mitogen for pre-B cells. SDF-1 has also been shown to be a chemokine which is chemotactic for different types of leukocytes. P-selectin, E-selectin, and L-selectin are cell adhesion molecules found in granules in endothelial cells and activated platelets and play a role in the recruitment of leukocytes to injury sites, particularly in vascular walls. Other examples of adhesion molecules include intracellular adhesion molecule (ICAM) and vascular adhesion molecule (VCAM). Nitric oxide synthases (NOS) catalyze the reaction whereby nitric oxide is synthesized from L-arginine. Different NOS isoforms, such as N-NOS, E-NOS, and I-NOS, are expressed in various tissue types, including endothelial cells, endocardial cells, and cardiomyocytes. Vascular endothelial growth factor (VEGF) is a signaling protein involved in both vasculoneogenesis and angiogenesis. In vitro, VEGF has been shown to stimulate endothelial cell mitogenesis and cell migration. CXCR4, also called fusin, is an alpha-chemokine receptor specific for SDF-1. Matrix metalloproteinases (MMPs) are zinc-dependant endopeptidases capable of degrading extracellular matrix proteins and also processing a number of bioactive molecules. MMPs are also believed to play a role on cell behaviors, such as cell proliferation, migration (adhesion/dispersion), differentiation, angiogenesis, apoptosis, and host defense. Connective tissue growth factor (CTGF) is a profibrotic factor, which is implicated in fibroblast proliferation, angiogenesis, and extracellular matrix (ECM) synthesis. Still further examples of proteins that can be used as signaling factors for vascular progenitor cells according to the present invention include angiogenin, angiopoietin-1, del-1, fibroblast growth factors (e.g., acidic (aFGF) and basic (bFGF)), follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, and insulin-like growth factors (IGFs). All of the foregoing examples, and combinations thereof, can be used in the recruitment of vascular progenitor cells according to the invention.

The present invention harnesses the signaling properties of chemokines (and other factors, such as listed above) found to be important in the recruitment of vascular progenitor cells and incorporates them into compositions and devices that can be used to remediate damaged vasculature. For example, the factors can be incorporated into a polymer composition, which itself can be used alone or can be coated onto existing devices, such as aneurysm coils and carotid stents. Thereby, the invention facilitates the body's own natural mechanism for vascular repair and even establishes an intact endothelium in certain instances.

The polymer compositions provided according to certain embodiments of the invention can comprise any type of polymer capable of use in the human body (i.e., biocompatible polymers) and capable of maintaining and delivering factors, such as described above, to a vascular site in need of repair. In preferred embodiments, the polymer used comprises a biodegradable polymer. In further embodiments, the polymer used comprises a polymer capable of use in forming a device, such as scaffolding or a stent.

For example, poly(lactide-co-glycolide) (PLGA) is a biodegradable polymer demonstrated to promote a consistent cellular reaction both in vitro and in vivo by slow acid release during its degradation, and locally serve as a potent chemokine for tissue macrophages and fibroblasts. PLGA is synthesized by means of random ring-opening co-polymerization of two different monomers, the cyclic dimers (1,4-dioxane-2,5-diones) of glycolic acid and lactic acid. During polymerization, successive monomeric units (of glycolic or lactic acid) are linked together in PLGA by ester linkages, thus yielding a linear, aliphatic polyester as a product. Depending on the ratio of lactide to glycolide used for the polymerization, different forms of PLGA can be obtained: these are usually identified in regard to the ratio of the monomer used (e.g., PLGA 75:25 identifies a copolymer whose composition is 75% lactic acid and 25% glycolic acid. PLGA for use in the present invention can have lactic acid to glycolic acid monomeric ratios in the range of 80:20 to 20:80, 75:25 to 25:75, 60:40 to 40:60, or 50:50.

Although PLGA is particularly useful, the invention should not be limited thereto but rather encompasses a variety of polymers. For example, non-degradable polymers, such as polyethylene terephthalate (PET) (e.g., DACRON®), polyethylene naphthalate (PEN), and polytetrafluoroethylene (PTFE), can also be used according to the invention. Preferably, when non-biodegradable polymers are used, such polymers are modified to comprise surface immobilized or controlled release biochemical attractors. Likewise, a variety of porous structures, such as polyvinyl acetate copolymers, can be used as controlled release systems for biochemical attractors. Such polymers can further be surface modified or otherwise formed to release antithrombotic agents to assist their function. Still further, hydrogel polymers could also be used according to the invention. Examples of specific further polymers that could be used include fibrin polymer and collagen polymers, as well as combinations of degradable and non-degradable polymers. Still other polymer compositions useful according to the invention include oxidized cellulose, crosslinked protein (e.g., casein or albumin), alginate, and polyhydroxyethyl methacrylate hydrogels.

In one embodiment, the polymer for use according to the invention comprises a polymer that is in a flowable state prior to placement within a subject (e.g., injection into a blood vessel) but that at least partially solidifies after placement within a subject. Such a polymer composition would be useful as a self-forming device for use according to the invention. In specific embodiments, the polymer could comprise a composition that solidifies upon contact with an ionic medium (such as blood). For example, Onyx liquid embolic material is an ethylene vinyl alcohol copolymer dissolved in dimethyl sulfoxide (DMSO) opacified with tantalum powder. Once coming into contact with an ionic solution, the DMSO dissipates and the Onyx solidifies into a spongy, cohesive material. This substance can be delivered to an aneurysm via a microcatheter once the neck of the aneurysm is temporarily occluded by a balloon which reduces the risk of the copolymer exiting the aneurysm and entering the native circulation. Similar materials according to the present invention can be prepared including the signaling factors useful for recruitment of vascular progenitor cells.

In another example, the polymer could comprise a composition that is flowable at room temperature (or a temperature below room temperature) but at least partially solidifies at an elevated temperature, such as human, or other animal, body temperature (e.g., at least about 30° C., at least about 35° C., or at least about 38° C.). In yet another example, the polymer composition could comprise multiple components that are mixed immediately prior to (or during) placement in the subject such that mixing of the components causes the polymer to at least partially solidify after a certain amount of time. Of course, other types of polymer compositions capable of transitioning from flowable to solid could be used according to the invention.

The polymer of the invention preferably incorporates signaling factors, such as those described above, and can be used in a variety of compositions, devices, and methods. For example, the polymer can incorporate one or more factors for immediate or controlled-release at a site of injury. In other embodiments, the polymer can incorporate the factor as DNA for gene delivery. As such, the polymer generally functions to promote the attachment of migrating vascular progenitor cells. Further, the polymer can be coated onto a variety of devices for use in vascular repair. Moreover, the polymer also serves as a suitable scaffolding by itself for the growth of vascular cells.

The invention particularly encompasses embodiments wherein a polymer as described herein is combined with vascular progenitor cells. Any method of combining cells with a polymer could be used. For example, the cells could be mixed with the polymer to form a substantially homogeneous composition. In other embodiments, the polymer could be formed into a pre-defined shape having a surface, and the cells could be attached (such as by covalent bonding, or other bonding, or by physical interactions) to the surface. Such compositions can also include further components, such as the factors described herein.

In certain embodiments, the invention provides a device useful in the treatment of vascular injury. Any device typically used in such treatment could be used according to the invention. For example, such devices can include coronary stents, peripheral vascular stents, carotid stents, aortic stent grafts, and intracranial stents. The polymer can be used to coat all or part of the device, as may be beneficial for the vascular repair. For example, only the exterior of a stent could be coated to facilitate cell growth at the vascular wall. Furthermore, any device capable of use in contact with blood flow (particularly devices designed for long-term residence in contact with blood flow) can be used according to the invention. Specific, non-limiting examples of such devices include pacemaker leads, electrodes, myocardial patches, heart valves, and the like.

Coating of a device with a polymeric material according to the invention can be via a single coating or multiple coatings. When a biodegradable polymer, such as PLGA is used as the coating material, the number of coatings used (as well as polymer concentration) can alter the effective lifetime of the coating (i.e., the amount of time the associated factors are present to recruit vascular progenitor cells). This is further illustrated below in Example 1. As seen therein, the inventive polymer can beneficially be altered to optimize the attachment of vascular progenitor cells and allow the polymer to serve as an optimal scaffolding for vascular cell growth. In certain embodiments, devices according to the invention can comprise one coating, two coatings, three coatings, four coatings, five coatings, six coatings, seven coatings, eight coatings, nine coatings, or ten coatings of the polymeric coating material incorporating signaling factors, as described herein. In specific embodiments, even more coatings could be used. The number of coatings of the coating material is only limited by the length of duration of factor release desired. The number of coatings needed to obtain a desired length of release can be easily determined by a skilled person without undue experimentation (such as illustrated in Example 1).

The polymer material for coating the surface of a device can be prepared as a solution using an aqueous solvent (e.g., water) or an organic solvent (e.g., chloroform, such as when using PLGA). Accordingly, the concentration of the polymer solution can vary with the type of polymer used. When PLGA is used as the polymer, a polymer solution having a concentration of up to about 50% by weight can be used. In further embodiments, the PLGA solution can have a concentration of up to about 40%, up to about 30%, up to about 20%, up to about 10%, up to about 8%, up to about 5%, or up to about 2% by weight.

Devices for use in the present invention can be formed of a variety of materials, including polymeric materials, as well as metallic materials (e.g., magnesium). Preferably, a device for use in the invention is formed of a material suitable for coating with a polymeric material and that will not disrupt the activity of factors present in the inventive polymer. In certain embodiments, a device can be modified prior to application of the inventive polymer, such as by grafting onto the surface thereof a material more suitable for coating with the inventive polymer. For example, in one embodiment, polyester materials such as DACRON®, can be used. Accordingly, it is possible for the device to be a combination of materials (e.g., a DACRON®-nitinol stent graft). In other embodiments, the devices for coating according to the invention can comprise other types of materials, such as platinum, nitinol, and hydrogel material.

The polymeric coating with the signaling factors incorporated therein can form a coating on existing devices by a variety of methods depending upon the type of material being coated and the type of polymer used in the coating. For example, in some embodiments, the polymeric coating material can be physically entrapped between fibers or within pores at the surface of the material being coated. In other embodiments, the polymer coating material can form covalent bonds with functional groups on the surface of the material being coated.

The inventive polymer can be coated onto a device by a variety of method. For example, the polymer can be applied “wet” by brushing, dipping, spraying, or the like and allowed to dry. In further embodiments, the inventive polymer can contain reactive groups capable of reacting with groups on the surface of the device to be coated, and the inventive polymer can attach to the surface of the device via covalent bonds formed between the inventive polymer and the device surface. In such embodiments, coating methods can include derivatizing the surface of a device to form the reactive groups prior to application of the inventive polymer.

Current standard therapy for carotid atherosclerosis includes percutaneous balloon angioplasty and stent placement. There is a significant incidence, however, of in-stent restenosis, occurring in 6%-16% of carotid stent and angioplasty patients. Vascular repair with re-endothelialization has been shown to be critical in preventing intimal thickening and in-stent restenosis after stent placement. The present invention is particularly useful in a technique for coating standard stents, such as nitinol stents, with the inventive polymer. Such coated stents are effective for promoting vascular progenitor cell attachment and vascular cell growth.

Other types of devices that could be at least partially coated with the polymer of the invention include devices for the treatment of intracranial aneurysms. Current standard therapy for intracranial aneurysms includes embolization treatment with endovascularly-delivered platinum detachable coils. However, aneurysm re-growth occurs in 21-34% of coil-treated aneurysms. The formation of neo-endothelium across the orifice of the coil-treated aneurysm seems to be critical in preventing aneurysm recurrence. The present invention specifically provides techniques for coating standard platinum aneurysm coils with the inventive polymer. Such coated aneurysm coils are effective for promoting vascular progenitor cell attachment and vascular cell growth.

In certain embodiments, the devices of the invention can be prepared partially or completely from the polymeric material described herein incorporating the signaling factors. The polymeric materials of the invention could be used to form stents or coils for use as described above. In such embodiments, the polymeric material could comprise polymers that are non-degradable or degrade very slowly. For example, polycaprolactone is an elastic polymer that degrades slowly and can be useful in forming devices for use according to the invention. Polybutylacrylate is another example of a polymer that degrades very slowly (and may even be viewed as being non-degradable) that is useful according to this embodiment of the invention. The device could also be formed of a polymer that is degradable or degrades more rapidly, such as for use in formation of temporary scaffolding devices. Any of the polymeric materials described herein for incorporation of signaling factors could also be used for forming devices according to the invention.

Work in a murine aneurysm model has shown that circulating vascular progenitor cells migrate (or “home”) to a site of vascular injury (e.g., endothelial disruption) in an aneurysm. The present invention takes advantage of this natural tendency by activating the signaling pathways for the recruitment of vascular progenitor cells and enhancing their ability for vascular repair by establishing scaffolding for vascular cell growth.

Thus, the invention further includes various methods of treatment for facilitating vascular repair. In one embodiment, the invention provides for methods for signaling the recruitment the vascular progenitor cells at a site of vascular injury. In further embodiments, the invention provides for methods of enhancing vascular repair at a site of vascular injury. Such methods generally comprise providing at the site of vascular injury a composition comprising a polymer according to the invention incorporating signaling factors that signal the recruitment of vascular progenitor cells. The composition can be provided as a coating on a medical device, such as a stent or coil. The composition can also be provided alone, such as in the form of a medical device (such as a stent or coil) formed partially or entirely from the composition.

### EXPERIMENTAL

The present invention will now be described with specific reference to an example. The following example is not intended to be limiting of the invention and is rather provided as an exemplary embodiment.

### Example 1

### Associated pH Change with Multiple PLGA Coatings

The pH change with time in DACRON® grafts coated with PLGA was evaluated. Four devices were prepared by coating with 5% or 10% by weight PLGA with either 5 coatings or 10 coatings. The pH change of the PLGA (in sample-phosphate buffered solution) was evaluated at 1-day, 8-days, 21-days and 42-days post sample preparation. The pH changes are illustrated below in Table 1, wherein pH changes at 21 and 42 days correspond with a degradation of the PLGA coating on the graft material. An increase in pH value corresponds to an increase in the acidity of the sample.

TABLE 1

| Sample | PLGA | Number of | pH Change | | | |
|---|---|---|---|---|---|---|
| Number | Wt. % | Coatings | 1-Day | 8-Days | 21-Days | 42-Days |
| 1 | 5% | 5 | −0.02 | +0.04 | +0.80 | +0.80 |
| 2 | 5% | 10 | −0.03 | +0.05 | +0.65 | +1.25 |
| 3 | 10% | 5 | −0.01 | +0.04 | +0.50 | +1.35 |
| 4 | 10% | 8 | −0.05 | +0.04 | +0.45 | +1.45 |

Analysis of the above samples indicated the percentage of PLGA coating remaining relative the amount present at the time of initial coating. At 21-days post application, sample 1 maintained about 95%, sample 2 maintained about 70%, sample 3 maintained about 80%, and sample 4 maintained about 45% of their respective initial coatings. At 42-days post application, samples 1 and 3 each maintained about 30%, and samples 2 and 4 each maintained about 20% of their respective initial coatings. This indicates the coatings with the signaling factors incorporate therein can be applied for long-term release of the signaling factors at sites in need of repair.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A composition comprising a polymer incorporating signaling factors that signal the recruitment of vascular progenitor cells.

2. The composition of claim 1, wherein the polymer comprises a biodegradable polymer.

3. The composition of claim 1, wherein the polymer comprises poly(lactide-co-glycolide) (PLGA).

4. The composition of claim 1, wherein the signaling factors comprise one or more chemokines.

5. The composition of claim 1, wherein the signaling factors are selected from the group consisting of stromal cell-derived factor-1, P-selectin, E-selectin, L-selectin, E-NOS, I-NOS, ICAM, VCAM, VEGF, CXCR4, matrix metalloproteinases (MMPs), CTGF, angiogenin, angiopoietin-1, del-1, fibroblast growth factors (FGFs), follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, insulin-like growth factors (IGFs), and combinations thereof.

6. A device for use in vascular repair, the device comprising a polymer incorporating signaling factors that signal the recruitment of vascular progenitor cells.

7. The device of claim 6, wherein the device is at least partially coated with the polymer.

8. The device of claim 7, wherein the device is selected from the group consisting of stents and aneurysm coils.

9. The device of claim 6, wherein the device is at least partially formed from the polymer.

10. The device of claim 9, wherein the device is selected from the group consisting of scaffolds, stents, and aneurysm coils

11. The device of claim 6, wherein the polymer comprises poly(lactide-co-glycolide) (PLGA).

12. The device of claim 6, wherein the signaling factors comprise one or more chemokines.

13. The device of claim 6, wherein the signaling factors are selected from the group consisting of stromal cell-derived factor-1, P-selectin, E-selectin, L-selectin, E-NOS, I-NOS, ICAM, VCAM, VEGF, CXCR4, matrix metalloproteinases (MMPs), CTGF, angiogenin, angiopoietin-1, del-1, fibroblast growth factors (FGFs), follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, insulin-like growth factors (IGFs), and combinations thereof.

14. A method for signaling the recruitment the vascular progenitor cells at a site of vascular injury comprising providing at the site of vascular injury a composition comprising a polymer incorporating signaling factors that signal the recruitment of vascular progenitor cells.

15. The method of claim 14, wherein the method further comprises providing a device selected from the group consisting of stents and aneurysm coils at least partially coated with the polymer.

16. The method of claim 14, wherein the polymer is in the form of a scaffold, stent, or aneurysm coil.

17. The method of claim 14, wherein the polymer comprises poly(lactide-co-glycolide) (PLGA).

18. The method of claim 14, wherein the signaling factors comprise one or more chemokines.

19. The method of claim 14, wherein the signaling factors are selected from the group consisting of stromal cell-derived factor-1, P-selectin, E-selectin, L-selectin, E-NOS, I-NOS, ICAM, VCAM, VEGF, CXCR4, matrix metalloproteinases (MMPs), CTGF, angiogenin, angiopoietin-1, del-1, fibroblast growth factors (FGFs), follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, insulin-like growth factors (IGFs), and combinations thereof.
